(19) United States
(12) Patent Application Publication
(10) Pub. No.: US 2021/0052436 A1
(43) Pub. Date: Feb. 25, 2021

(54) WETNESS INDICATING COMPOSITIONS INCLUDING AN AMPHIPHILIC POLYDIACETYLENE AND ABSORBENT ARTICLES INCLUDING THE SAME

(71) Applicant: Kimberly-Clark Worldwide, Inc., Neenah, WI (US)

(72) Inventor: Xuedong Song, Aplharetta, GA (US)

(21) Appl. No.: 16/963,567

(22) PCT Filed: Jan. 26, 2018
(86) PCT No.: PCT/US2018/015456

§ 371 Date: Jul. 21, 2020

(51) Int. Cl. A61F 13/42; A61F 13/49; A61L 15/52; A61L 15/56

(52) U.S. Cl. CPC A61F 13/42; A61F 13/49007; C08L 49/00; A61L 15/56; A61F 2013/422; A61L 15/52

(57) ABSTRACT

A wetness indicating composition can include a mixing solvent including water and a water-miscible volatile organic solvent. The wetness indicating composition can also include an amphiphilic polydiacetylene and a hydrophobic binder. In some embodiments, the wetness indicating composition can be applied to a substrate and serve as a wetness indicator for an absorbent article.

### TECHNICAL FIELD

The present disclosure relates to wetness indicating compositions. More specifically, the present disclosure relates to wetness indicating compositions to be utilized in absorbent articles.

### BACKGROUND OF THE DISCLOSURE

Many products, including consumer, health care, and professional products, are more effectively used by consumers when containing a feature that can communicate or indicate to the user that the product is wetted with an aqueous solution (e.g., urine). For example, wetness sensing capability in a personal hygiene article, such as an absorbent article, has been a desirable and welcome feature. Disposable absorbent articles such as diapers, training pants, incontinence pads, and the like are highly absorbent and efficiently pull moisture away from the wearer, thereby reducing skin irritation caused by prolonged wetness exposure. However, because these articles are so absorbent, wearers may not realize they have urinated, particularly if they are inexperienced toddlers who may not recognize the meaning of body sensations associated with urination. Thus, the wearer may not recognize their urination control failure or be aware the article should be changed. Furthermore, caregivers may not recognize that the absorbent article requires changing.

Visual mechanisms have been employed to indicate the presence of wetness in absorbent articles. There are a large number of wetness sensing technologies that currently exist including electronic-based wetness sensors, color-based wetness sensors, and enzyme-based wetness sensors. However, all those wetness sensing technologies are not ideal and have one or more limitations. For instance, the electronic based wetness sensors are generally too expensive to be disposable, while enzyme-based wetness sensors may have stability issues. Water-soluble dye-based wetness indicators experience dye leaching upon wetting and can present blurred graphics. Additionally, some water-soluble dye-based wetness indicators may be limited in the initial starting color and/or the color of the ending color after indicating wetness.

Thus, a need exists for a wetness indicating composition that is cost effective, stable and capable of a different variety of hue changes discernable by a wearer and/or caregiver. A personal hygiene article, in particular an absorbent article, which incorporates a substrate having such a wetness indicating composition would be beneficial as well.

### SUMMARY OF THE DISCLOSURE

In one embodiment, a wetness indicating composition is provided. The wetness indicating composition can include a mixing solvent including water and a water-miscible volatile organic solvent. The wetness indicating composition can also include an amphiphilic polydiacetylene. The wetness indicating composition can further include a hydrophobic binder.

In another embodiment, a substrate for indicating wetness is provided. The substrate can include a basesheet. The substrate can also include a wetness indicating composition applied to a hydrophobic surface of the basesheet. The wetness indicating composition can include a mixing solvent comprising water and a water-miscible volatile organic solvent. The wetness indicating composition can further include an amphiphilic polydiacetylene and a hydrophobic binder.

In yet another embodiment, a method of indicating wetness in an absorbent article is provided. The absorbent article can include a body-side liner, an outer cover, and an absorbent body disposed between the body-side liner and the outer cover. The method can include providing the absorbent article. The absorbent article can include a hydrophobic surface. The method can further include applying a wetness indicating composition to the hydrophobic surface. The wetness indicating composition can include a mixing solvent comprising water and a water-miscible volatile organic solvent. The wetness indicating composition can further include an amphiphilic polydiacetylene and a hydrophobic binder. The wetness indicating composition can be configured to change color from a first hue to a second hue upon being wetted to indicate wetness in the absorbent article.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is a front perspective view of an exemplary embodiment of an absorbent article, such as a diaper, including a wetness indicating composition of the present disclosure, where the article is in an unfastened, relaxed condition.

FIG. 2 is a top plan view of the absorbent article of FIG. 1 in a stretched, laid flat, unfastened condition.

FIG. 3 is a cross-sectional view taken along line 3-3 from FIG. 2, except with the absorbent article being in a relaxed condition such that the containment flaps extend away from the absorbent body.

Repeat use of reference characters in the present specification and drawings is intended to represent the same or analogous features or elements of the disclosure.

### DETAILED DESCRIPTION OF THE DISLOSURE

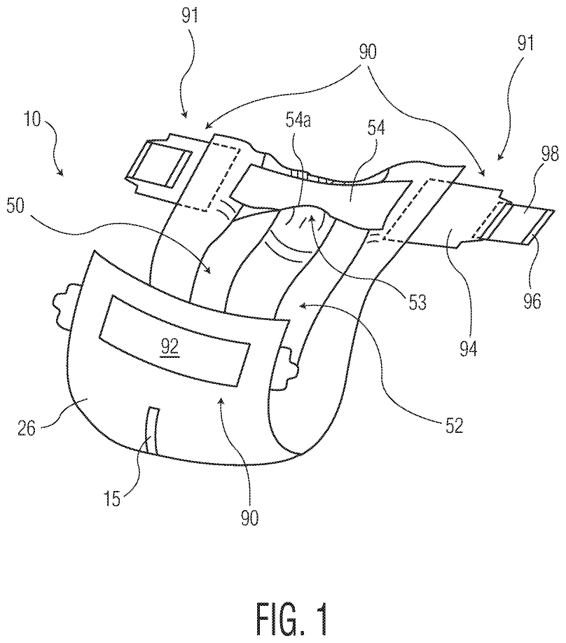

In an embodiment, the present disclosure is generally directed towards wetness indicating compositions that include amphiphilic polydiacetylene (APDA). The wetness indicating compositions can from part of a wetness indicator 15 and can be utilized on a substrate within or forming a part of an absorbent article 10, such as a diaper depicted in FIGS. 1-3 herein. Alternatively, the wetness indicating compositions can be utilized in various other applications. The wetness indicating compositions can provide the benefits of a visually discernable indication of wetness to a user, as well as a different variety of hues than are currently commercially available. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

Definitions:

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hue” refers to colors such as red, yellow, green, and blue. Different hues are caused by absorption of different wavelengths of light.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

Wetness Indicating Composition

The wetness indicating composition of the present disclosure can include a mixing solvent and amphiphilic polydiacetylene (APDA). APDA has a relatively regular conjugated structure and provides a blue hue.

The mixing solvent can include water and a water-miscible volatile organic solvent. In some embodiments, the water-miscible volatile organic solvent can include, but is not limited to, volatile low alcohols, such as ethanol, propanol, and butanol. Other volatile organic solvents can include acetone, ethyl acetate, tetrahydrofuran, isopropanol, and acetonitrile. Preferred water-miscible volatile organic solvents can include methanol, ethanol, propanol, acetone, butanone, and tetrahydrofuran. In some embodiments, the mixing solvent can provide from about 40 wt. % to about 95 wt. % of the total weight of the wetness indicating composition, in some embodiments, from about 50 wt. % to about 90 wt. %, and in some embodiments, from about 60 wt. % to about 70 wt. % by total weight of the wetness indicating composition. In preferred embodiments, water comprises at least about 60% by weight of the mixing solvent. The water-miscible volatile organic solvent can provide the functionality of evaporating after the wetness indicating composition is applied to a substrate.

The wetness indicating compositions can also include a hydrophobic binder. The hydrophobic binder helps to provide enhanced stability of the wetness indicating composition to remain on the substrate to which it is applied. The hydrophobic binder can also help to adjust viscosity of the composition that is amicable for printing. In the experimentation documented later herein, it was surprisingly discovered that the wetness indicating composition including APDA had good stability on certain substrates. Preferred examples of the hydrophobic binder can include, but are not limited to, cellulose derivatives, homopolymers and copolymers of polyacrylates, polyurethanes, and polyesters. In some embodiments, the hydrophobic binder can form from about 0.1 wt. % to about 20.0 wt. %, or from about 0.5 wt. % to about 10.0 wt. %, or from about 2.0 wt. % to about 5.0 wt. % of the wetness indicating composition (by total weight of the wetness indicating composition).

A stabilizer may form another component of the wetness indicating composition in some embodiments. Stabilizers can help to prevent precipitation of components or layer separation within the wetness indicating composition. One desirable stabilizer is hydroxypropyl-modified guar gum. Other suitable stabilizers include gum arabic, guar gum, xanthan gum, hydroxyethyl cellulose, methyl cellulose, ethyl cellulose, starches, casein, gelatin, and water-soluble polymers such as polyvinyl alcohol and styrene-maleic anhydride copolymer salt. The stabilizer, if present, can form from about 0.01 wt. % to about 10.0 wt. %, and more preferably from about 1.0 wt. % to about 5.0 wt. % of the wetness indicating composition (by total weight of the wetness indicating composition).

The wetness indicating composition can optionally include a wettability modifier to help improve its ability to be applied and adhered to a substrate. Exemplary wettability modifiers can include, but are not limited to, a surfactant (e.g., nonionic, cationic, anionic, or zwitterionic) or a mixture of surfactants. The surfactants may also help enhance the sensitivity and contrast provided by the wetting indicating composition. Particularly desired surfactants are nonionic surfactants, such as ethoxylated alkylphenols, ethoxylated and propoxylated fatty alcohols, ethylene oxide-propylene oxide block copolymers, ethoxylated esters of fatty (C8-C18) acids, condensation products of ethylene oxide with long chain amines or amides, condensation products of ethylene oxide with alcohols, acetylenic diols, and mixtures thereof. Various specific examples of suitable nonionic surfactants include, but are not limited to, methyl gluceth-10, PEG-20 methyl glucose distearate, PEG-20 methyl glucose sesquistearate, C11-C15 pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C.sub.6-C.sub.22) alcohol, including 3 to 20 ethylene oxide moieties, polyoxyethylene-20 isohexadecyl ether, polyoxyethylene-23 glycerol laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG 600 dioleate, PEG 400 dioleate, and mixtures thereof. Commercially available nonionic surfactants may include the SURFYNOL® range of acetylenic diol surfactants available from Air Products and Chemicals of Allentown, Pa. and the TWEEN® range of polyoxyethylene surfactants available from Fischer Scientific of Pittsburgh, Pa. While the wetness indicating composition may be generally free of such wettability modifiers, it should of course be understood that a small amount may still be present in the resulting composition. Regardless, the wettability modifier can typically form an amount less than about 5.0 wt. %, in some embodiments less than about 2.0 wt. %, and in some embodiments, from about 0.01 wt. % to about 1.0 wt. % of the wetness indicating composition (by total weight of the wetness indicating composition).

In some embodiments, the wetness indicating composition can include an adhesion promoter. Exemplary adhesion promoters can include, but are not limited to, nitrocellulose, polyvinyl butyral, cellulose acetate butyrate, polyamide, and polyurethane. In embodiments including an adhesion promoter, the adhesion promoter can comprise from about 1.0 wt. % to about 15.0 wt. % of the wetness indicating composition (by total weight of the wetness indicating composition).

The wetness indicating composition can indicate the presence of wetness through a visually discernable color change. For example, the wetness indicating composition can provide a first hue to a second hue upon being wetted. As discussed above, APDA in the wetness indicating composition can provide a first hue of blue. Depending on the other components of the wetness indicating composition, the second hue can be pink or purple. The extent of the color change is generally sufficient to provide a “real-time” indication of wetness on the substrate by the human eye. This color change may, for example, be represented by a certain change in the absorbance reading as measured using a conventional test known as “CIELAB”, which is discussed in Pocket Guide to Digital Printing by F. Cost, Delmar Publishers, Albany, N.Y. ISBN 0-8273-7592-1 at pages 144 and 145. This method defines three variables, L*, a*, and b*, which correspond to three characteristics of a perceived color based on the opponent theory of color perception. The three variables have the following meaning:

L*=Lightness (or luminosity), ranging from 0 to 100, where 0=dark and 100=light;

a*=Red/green axis, ranging approximately from −100 to 100; positive values are reddish and negative values are greenish; and

b*=Yellow/blue axis, ranging approximately from −100 to 100; positive values are yellowish and negative values are bluish.

Because CIELAB color space is somewhat visually uniform, a single number may be calculated that represents the difference between two colors as perceived by a human. This difference is termed [symbol] E and calculated by taking the square root of the sum of the squares of the three differences (L*, [symbol] a*, and [symbol] b*) between the two colors. In CIELAB color space, each [symbol] E unit is approximately equal to a “just noticeable” difference between two colors. CIELAB is therefore a good measure for an objective device-independent color specification system that may be used as a reference color space for the purpose of color management and expression of changes in color. Using this test, color intensities (L*, a*, and b*) may thus be measured using, for instance, a handheld spectrophotometer from Minolta Co. Ltd. of Osaka, Japan (Model # CM2600d). This instrument utilizes the D/8 geometry conforming to CIE No. 15, ISO 7724/1, ASTME1164 and JIS Z8722-1982 (diffused illumination/8-degree viewing system. The D65 light reflected by the specimen surface at an angle of 8 degrees to the normal of the surface is received by the specimen-measuring optical system. Typically, the color change is represented by a ΔE of about 2 or more, in some embodiments about 3 or more, and in some embodiments, from about 5 to about 50.

The wetness indicating composition can be applied to a basesheet to form a wetness indicator 15. The basesheet preferably includes a hydrophobic surface to which the wetness indicating composition can be applied. It is preferable that the hydrophobic surface has a surface tension of less than 60 dynes/cm, or more preferably, less than 45 dynes/cm.

In one embodiment, a process for making a wetness indicator 15 can include polymerizing diacetylene monomers in well-organized form, such as self-assemblies. Examples of amphiphilic diacetylenes include, but are not limited to, diacetylene fatty acids, diacetylene salts of metals and imidazolium containing diacetylenes. The process can include dissolving such amphiphilic diacetylene monomers in water. The process can also include adding water-miscible organic solvents to the diacetylene solution to make self-assembled diacetylene. The process can then include applying the self-assembled diacetylene solution on a hydrophobic surface of a basesheet. The process can then include drying the diacetylene layer applied to the basesheet. Finally, the process can include applying ultra-violet (UV) light to the diacetylene layer on the basesheet to polymerize the diacetylene layer to form an amphiphilic polydiacetylene, or APDA, layer. In other embodiments, the process can include applying the UV light to the amphiphilic diacetylene monomers to provide APDA in solution prior to applying such APDA mixture to the substrate and drying such APDA. Preferably, the UV light used to polymerize the diacetylene layer can range from about 150 nm to about 350, and more preferably from about 200 nm to about 280 nm, and can be of various intensities.

Experimental Wetness Indicating Compositions

Experiment 1: Preparation of diacetylene self-assemblies

0.433 g of 10, 12-tricosadiynoic acid (TCDA) was dissolved in 2.4 mL tetrahydrofuran. 70 mg of potassium hydroxide was dissolved in 0.1 mL water. The potassium hydroxide solution was then dropped into the TCDA solution under stirring. The mixture was stirred for one hour to make a solution of TCDA sodium salt (TCDA-Na) self-assemblies.

Experiment 2: Application and polymerization of diacetylene self-assemblies to substrate

The solution prepared on Experiment 1 was applied by a brush on different substrates ranging from polypropylene (“PP”) film, hydroknit sheet, printing paper, KIMTECH SCIENCE® KIMWIPES, spunbond sheet, and spunbond-meltblown-spunbond (“SMS”) sheet to make a thin layer of the solution and was dried in fume hood of oven at 60° C. for a few minutes. A hand-held UV lamp providing UV light at a wavelength of 265nm was used to irradiate the Cana layer on the various substrates for a few seconds to polymerize the TCDA-Na. All the colorless TCDA-Na layers became blue almost immediately upon polymerization in forming a poly(TCDA-Na) layer on the respective substrates. It was observed that the polydiacetylene layers had good adhesion on the samples of PP film, spunbond sheet, and SMS sheet, which all provide a hydrophobic surface, however, the adhesion of the polydiacetylene layers on the samples of hydroknit, printing paper, and KIMTECH SCIENCE® KIMWIPES were not as strong.

Experiment 3: Wetness testing of sample wetness indicators

A drop of water or synthetic urine was applied to the PDA layer on the substrates of Experiment 2. The synthetic urine used for Experiments herein included water, urea, sodium choloride, magnesium sulfate, and calcium chloride and is commercially available from Ricca Chemical Compnay. For the poly(TCDA-Na) layers on the substrates of the PP film, spunbond, and SMS substrates, the blue color changes from blue to pink or purple upon contact with a drop of water or synthetic urine. However, no color change was observed when the poly(TCDA-Na) layer on the substrates of the hydroknit sheet, printing paper, or KIMTECH SCIENCE® KIMWIPES.

Experiment 4: Preparation of diacetylene self-assemblies

0.933 g TCDA and 0.3 g cellulose acetate propriate (CAP) was dissolved in 4.8 mL tetrahydrofuran. 140 mg potassium hydroxide was dissolved in 0.1 mL water. The potassium hydroxide solution was dropped into TCDA/CAP solution under stirring. The mixture was stirred for one hour to make a TCDA-Na self-assemblies solution.

Experiment 5: Wetness testing of sample wetness indicators

The TCDA-Na self-assemblies solution of Experiment 4 were applied to the same substrates as noted above in Experiment 2. The TCDA-Na layers were dried and UV light was applied to such TCDA-Na layers in the same methodology as described above in Experiment 2 to provide poly(TCDA-Na) layers on the substrates. It was noted that for the poly(TCDA-Na) layers on the substrates of the PP film, spunbond, and SMS substrates, the blue color changes from blue to pink or purple upon contact with a drop of water or synthetic urine. However, no color change was observed when the poly(TCDA-Na) layer on the substrates of the hydroknit sheet, printing paper, or KIMTECH SCIENCE® KIMWIPES.

Experiment 6: Alternative polymerization of diacetylene monomers to form sample wetness indicators

0.433 g TCDA was dissolved in 2.4 mL tetrahydrofuran. 70 mg potassium hydroxide was dissolved in 0.1 mL water. The potassium hydroxide solution was dropped into the TCDA solution under stirring. This resultant solution was stirred for one hour to make a TCDA-Na self-assemblies solution. The mixture was then irradiated with a UV lamp at 265 nm for a short period of time (from a few seconds to minutes) to make a blue poly(TCDA-Na) suspension. The poly(TCDA-Na) suspension was applied to the same substrates as noted above in Experiment 2 and dried to form a layer of poly(TCDA-Na) according to the methodology of Experiment 2. Upon contact with a drop of water or synthetic urine, the blue PDA layers changed color from blue to pink or purple on hydrophobic substrates depending upon the irradiation time. The poly(TCDA-Na) produced by low level of irradiation (about 1 second to about 20 seconds) changes to pink upon wetting while the poly(TCDA-Na) produced by higher level of irradiation (about 30 seconds to about 120 seconds) changes to purple. As noted in Experiments 3 and 5, the blue PDA on hydrophilic substrates of the hydroknit sheet, printing paper, and KIMTECH SCIENCE® KIMWIPES showed no color change upon contact with water or synthetic urine. This provided an alternative polymerization method of the diacetylene monomers to provide the PDA suspension to be capable of producing an effective wetness indicator on the hydrophobic substrates.

Absorbent Article:

In one embodiment, the wetness indicating composition as described herein can be used on a substrate in, or that forms part of, an absorbent article 10. Referring to FIGS. 1-3, a non-limiting illustration of an absorbent article 10, for example, a diaper, including a wetness indicating composition of the present disclosure is illustrated. Other embodiments of the absorbent article could include training pants, youth pants, adult incontinence garments, and feminine hygiene articles. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure.

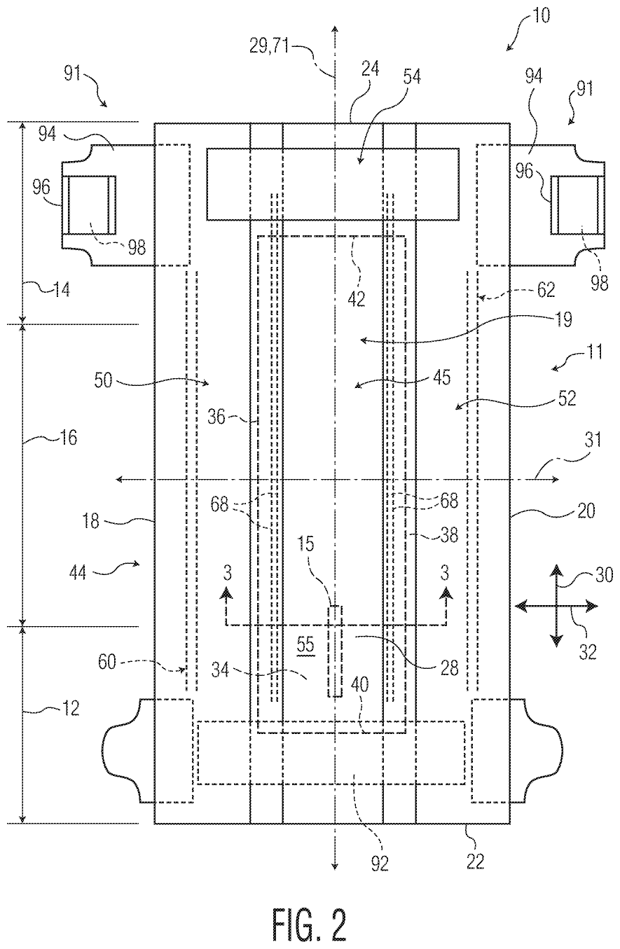

The absorbent article 10 illustrated in FIGS. 1 and 2 can include a chassis 11. The absorbent article 10 can include a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front end region, the rear waist region 14 can be referred to as the rear end region, and the crotch region 16 can be referred to as the intermediate region.

The absorbent article 10 can have a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIG. 2. In other embodiments, the longitudinal side edges 18, 20 can be curved between the front waist edge 22 and the rear waist edge 24.

The front waist region 12 can include the portion of the absorbent article 10 that, when worn, is positioned at least in part on the front of the wearer while the rear waist region 14 can include the portion of the absorbent article 10 that, when worn, is positioned at least in part on the back of the wearer. The crotch region 16 of the absorbent article 10 can include the portion of the absorbent article 10 that, when worn, is positioned between the legs of the wearer and can partially cover the lower torso of the wearer. Portions of the longitudinal side edges 18, 20 in the crotch region 16 can generally define leg openings for the legs of the wearer when the absorbent article 10 is worn.

The absorbent article 10 can include an outer cover 26 and a bodyside liner 28. The outer cover 26 and the bodyside liner 28 can form a portion of the chassis 11. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIG. 2, the absorbent article 10 can have a longitudinal axis 29 extending in the longitudinal direction 30 and a lateral axis 31 extending in the lateral direction 32.

The chassis 11 can include an absorbent body 34. The absorbent body 34 can be disposed between the outer cover 26 and the bodyside liner 28. The absorbent body 34 can have longitudinal edges, 36 and 38, which, in an embodiment, can form portions of the longitudinal side edges, 18 and 20, respectively, of the absorbent article 10. The absorbent body 34 can have a first end edge 40 that is opposite a second end edge 42, respectively, which, in an embodiment, can form portions of the waist edges, 22 and 24, respectively, of the absorbent article 10. In some embodiments, the first end edge 40 can be in the front waist region 12. In some embodiments, the second end edge 42 can be in the rear waist region 14. In an embodiment, the absorbent body 34 can have a length and width that are the same as or less than the length and width of the absorbent article 10. The bodyside liner 28, the outer cover 26, and the absorbent body 34 can form part of an absorbent assembly 44. In some embodiments, if a fluid transfer layer 46 is present, the acquisition layer 48 can be between the bodyside liner 28 and the fluid transfer layer 46 as is known in the art. The absorbent assembly 44 can also include a spacer layer (not shown) disposed between the absorbent body 34 and the outer cover 26 as is known in the art. The absorbent assembly 44 can include other components in some embodiments. It is also contemplated that some embodiments may not include a fluid transfer layer 46, and/or an acquisition layer 48, and/or a spacer layer.

The absorbent article 10 can be configured to contain and/or absorb liquid, solid, and semi-solid body exudates discharged from the wearer. In some embodiments, a pair of containment flaps 50, 52 can be configured to provide a barrier to the lateral flow of body exudates. In some embodiments, the absorbent article 10 can include a waist containment member 54. The waist containment member 54 can be disposed in the rear waist region 14 of the absorbent article 10, and/or in the front waist region 12 of the absorbent article 10.

The absorbent article 10 can further include leg elastic members 60, 62 as are known to those skilled in the art. The leg elastic members 60, 62 can be attached to the outer cover 26 and/or the bodyside liner 28 along the opposite longitudinal side edges, 18 and 20, and positioned in the crotch region 16 of the absorbent article 10. The leg elastic members 60, 62 can be parallel to the longitudinal axis 29 as shown in FIG. 2 or can be curved as is known in the art. The leg elastic members 60, 62 can provide elasticized leg cuffs.

Additional details regarding each of these elements of the absorbent article 10 described herein can be found below and with reference to the FIGS. 1-3.

Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

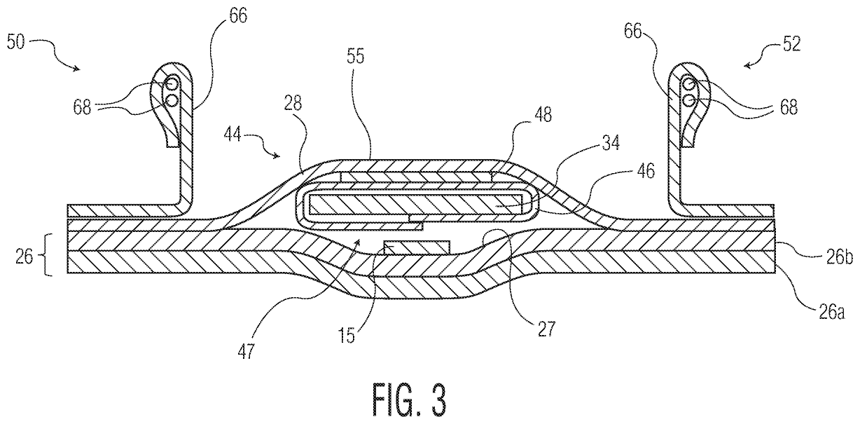

In preferred embodiments, the outer cover 26 includes at least one hydrophobic surface. In some embodiments, the outer cover 26 can provide a basesheet to which the wetness indicating compositions as described herein can be applied (as shown in FIG. 3) to provide a wetness indicator 15 for the absorbent article 10. In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two layer construction, including an outer layer 26a and an inner layer 26b which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer 26a of the outer cover 26 can be any suitable material and may be one that provides a generally cloth-like texture or appearance to the wearer, and can be generally referred to as a facing layer. An example of such material can be a 100% polypropylene bonded-carded web with a diamond bond pattern available from Sandler A.G., Germany, such as 30 gsm Sawabond 4185® or equivalent. Another example of material suitable for use as a facing layer 26a of an outer cover 26 can be a 20 gsm spunbond polypropylene non-woven web. The facing layer may 26a also be constructed of the same materials from which the bodyside liner 28 can be constructed as described herein.

The liquid impermeable inner layer 26b of the outer cover 26 (or the liquid impermeable outer cover 26 where the outer cover 26 is of a single-layer construction) can be either vapor permeable (i.e., “breathable”) or vapor impermeable. The inner layer 26b can be generally referred to as a film layer. The film layer 26b (or the liquid impermeable outer cover 26 where the outer cover 26 is of a single-layer construction) can be manufactured from a thin plastic film. The liquid impermeable inner layer 26b (or the liquid impermeable outer cover 26 where the outer cover 26 is of a single-layer construction) can inhibit liquid body exudates from leaking out of the absorbent article 10 and wetting articles, such as bed sheets and clothing, as well as the wearer and caregiver. Additionally, the film layer 26b of the outer cover 26 can have a wetness indicator 15 including a wetness indicating composition as described further herein. Preferably, the wetness indicating composition can be applied to the body facing surface 27 of the film layer 26b to form the wetness indicator 15, such that the wetness indicating composition is in closer proximity to the absorbent body 34 of the absorbent article 10.

In some embodiments, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10. The absorbent body 34 can have a length and width that can be less than or equal to the length and width of the absorbent article 10.

In an embodiment, the absorbent body 34 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 34 can be a matrix of cellulosic fluff and superabsorbent material. In an embodiment, the absorbent body 34 may be constructed of a single layer of materials, or in the alternative, may be constructed of two or more layers of materials.

Various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent body 34 can be free of superabsorbent material.

If a spacer layer is present, the absorbent body 34 can be disposed on the spacer layer and superposed over the outer cover 26. The spacer layer can be bonded to the outer cover 26, for example, by adhesive. In some embodiments, a spacer layer may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In some embodiments, at least a portion of a layer, such as but not limited to, a fluid transfer layer 46 and/or a spacer layer, can be positioned between the absorbent body 34 and the outer cover 26. The absorbent body 34 can be bonded to the fluid transfer layer 46 and/or the spacer layer.

Bodyside Liner:

The bodyside liner 28 of the absorbent article 10 can overlay the absorbent body 34 and the outer cover 26 and can be configured to receive insults of exudates from the wearer and can isolate the wearer's skin from liquid waste retained by the absorbent body 34. The bodyside liner 28 can from at least a part of the body facing surface 19 of the chassis 11.

In various embodiments, a fluid transfer layer 46 can be positioned between the bodyside liner 28 and the absorbent body 34 (as shown in FIG. 3). In various embodiments, an acquisition layer 48 can be positioned between the bodyside liner 28 and the absorbent body 34 or a fluid transfer layer 46, if present (as shown in FIG. 3). In various embodiments, the bodyside liner 28 can be bonded to the acquisition layer 48, or to the fluid transfer layer 46 if no acquisition layer 48 is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 34 and/or a fluid transfer layer 46, if present, and/or an acquisition layer 48, if present, and/or a spacer layer, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. In some embodiments, the bodyside liner 28 and the outer cover 26 may be of the same dimensions in width and length. In some embodiments, however, the bodyside liner 28 may be narrower than the outer cover 26 and/or shorter than the outer cover 26. In some embodiments, the length of the bodyside liner 28 can range from 50%-100% of the length of the absorbent article 10 as measured in a direction parallel to the longitudinal axis 29. In some embodiments, the bodyside liner 28 can be of greater width than the outer cover 26. It is also contemplated that the bodyside liner 28 may not extend beyond the absorbent body 34 and/or may not be secured to the outer cover 26. In some embodiments, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material.

The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,474,660 invented by Kirby, Scott S.C. et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from about 10 or 12 to about 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral direction 32 of the absorbent article 10. In other aspects, the bodyside liner 28 can be stretchable, and more suitably elastic, in both the lateral and the longitudinal directions 32, 30, respectively.

Containment Flaps:

In an embodiment, the absorbent article 10 can include a pair of containment flaps 50, 52. The containment flaps 50, 52 can be formed separately from the absorbent chassis 11 and attached to the chassis 11 or can be formed integral to the chassis 11. In some embodiments, the containment flaps 50, 52 can be secured to the chassis 11 of the absorbent article 10 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an embodiment, the containment flaps 50, 52 can extend generally in a longitudinal direction 30 from the front waist region 12 of the absorbent article 10, through the crotch region 16 to the rear waist region 14 of the absorbent article 10. In some embodiments, the containment flaps 50, 52 can extend in a direction substantially parallel to the longitudinal axis 29 of the absorbent article 10, however, in other embodiments, the containment flaps 50, 52 can be curved, as is known in the art.

In embodiments where the containment flaps 50, 52 are coupled to the chassis 11, the containment flaps 50, 52 can be bonded to the bodyside liner 28, the outer cover 26, or another layer, such as a spacer layer, if present, with a barrier adhesive (not shown), as is known in the art. Of course, the containment flaps 50, 52 can be bonded to other components of the chassis 11 and can be bonded with other suitable means other than a barrier adhesive. For example, the containment flaps 50, 52 can be bonded to the bodyside liner 28, the outer cover 26, or another layer with pressure bonding, thermal bonding, or ultrasonic bonding. The containment flaps 50, 52 can be constructed of a fibrous material which can be similar to the material forming the bodyside liner 28. Other conventional materials, such as polymer films, can also be employed.

As illustrated in FIGS. 2 and 3, the containment flaps 50, 52 can each include a base portion 64 and a projection portion 66. The base portion 64 can be bonded to the chassis 11, for example, to the bodyside liner 28 or the outer cover 26 as mentioned above. The projection portion 66 can be configured to extend away from the body facing surface 19 of the chassis 11 at least in the crotch region 16 when the absorbent article 10 is in a relaxed configuration, as illustrated in FIGS. 1 and 3. In other words, the projection portion 66 can be configured to extend away from the absorbent body 34. The containment flaps 50, 52 can include one or more flap elastic members 68, such as the two flap elastic strands depicted in FIGS. 2 and 3. Suitable elastic materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastic properties itself.

The flap elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, when the absorbent article 10 is in a relaxed configuration.

It is contemplated that the containment flaps 50, 52 can be of various configurations and shapes and can be constructed by various methods, or can be omitted completely from the absorbent article 10 without departing from the scope of this disclosure.

Leg Elastics:

Leg elastic members 60, 62 can be secured to the outer cover 26, such as by being bonded thereto by laminate adhesive, generally laterally inward of the longitudinal side edges, 18 and 20, of the absorbent article 10. The leg elastic members 60, 62 can form elasticized leg cuffs that further help to contain body exudates. In an embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein in FIG. 2 each include two elastic strands. A wide variety of elastic materials may be used for the leg elastic members 60, 62. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some embodiments. Of course, the leg elastic members 60, 62 can be omitted from the absorbent article 10 without departing from the scope of this disclosure.

Waist Containment Member:

In an embodiment, the absorbent article 10 can have one or more waist containment members 54. FIGS. 1 and 2 illustrate a preferred embodiment of a waist containment member 54 on an absorbent article 10, such as a diaper. The waist containment member 54 can be disposed in the rear waist region 14, while in some embodiments, the waist containment member 54 can be disposed in the front waist region 12. The waist containment member 54 can be disposed on the body facing surface 45 of the absorbent assembly 44. In some embodiments, such as in embodiments illustrated in FIGS. 1-3, and 5, the waist containment member 54 can be disposed on the body facing surface 55 of the bodyside liner 28. The waist containment member 54 can be coupled to the chassis 11 such that a portion 54a of the waist containment member 54 is free to move with respect to the chassis 11 and can form a pocket 53 to help contain body exudates.

The waist containment member 54 can be comprised of a variety of materials. In a preferred embodiment, the waist containment member 54 can be comprised of a SMS material. However it is contemplated that the waist containment member 54 can be comprised of other materials including, but not limited to, a spunbond-film-spunbond (“SFS”), a bonded carded web (“BCW”), or any non-woven material. In some embodiments, the waist containment member 54 can be comprised of a laminate of more than one of these exemplary materials, or other materials. In some embodiments, the waist containment member 54 can be comprised of a liquid impermeable material. In some embodiments, the waist containment member 54 can be comprised of a material coated with a hydrophobic coating. In some embodiments, the waist containment member 54 can include an elastic material to provide additional fit and containment properties to the absorbent article 10. In such an embodiment, suitable elastic materials can include, but are not limited to, sheets, strands or ribbons of natural rubber, synthetic rubber, or thermoplastic elastomeric polymers. The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist containment member 54 may be omitted from the absorbent article 10 without departing from the scope of this disclosure.

Wetness Indicator:

In some embodiments, the wetness indicating composition of the present disclosure can form part of a wetness indicator 15 for an absorbent article 10, such as shown in FIGS. 1-3.

The wetness indicator 15 can be configured in various fashions on an absorbent article 10. The wetness indicating composition, as discussed above, can be applied to the basesheet that is inserted into, or forms part of, the absorbent article 10. For example, the wetness indicator 15 can be formed by applying a wetness indicating composition to a hydrophobic surface, such as a body facing surface 27 of the inner film layer 26b, as illustrated in FIG. 3.

In another embodiment, the wetness indicator 15 can be formed by applying a wetness indicating composition to a hydrophobic surface of the fluid transfer layer 46. In such an embodiment, it can be preferable to apply the wetness indicating composition to a portion 47 of the fluid transfer layer 46 disposed between the absorbent body 34 and the outer cover 26 to form a wetness indicator 15 for the absorbent article 10. Such a configuration provides improved visibility for a user to see the wetness indicator 15 while the absorbent article 10 is on a wearer.

In yet another alternative embodiment, the wetness indicating composition as described herein can form a wetness indicator 15 for an absorbent article 10 by being applied to a basesheet to form a discrete substrate that is then coupled to one or more of the components forming the absorbent article 10.

The wetness indicating composition can be applied to a basesheet, or material forming a component of the absorbent article 10, in various ways. For example, the wetness indicating composition can be sprayed, roll-coated, spin-coated, printed (including gravure, flexographic, and screen printing techniques), dipped, or applied to a basesheet or material forming a component of the absorbent article 10 in any other suitable way known to one of ordinary skill in the art. In some embodiments, it can be preferable to polymerize a diacetylene solution (as previously described) after such a solution is applied to the basesheet or material forming a component of the absorbent article 10 to provide an APDA composition that can serve as a wetness indicator 15.

The wetness indicating composition forming a wetness indicator 15 can be configured in a variety of shapes and/or locations on the absorbent article 10. In one embodiment, the wetness indicator 15 can resemble a discrete line segment, as illustrated in FIG. 2. A longitudinal axis 71 of the wetness indicator 15 can be generally aligned with the longitudinal axis 29 of the absorbent article 10. Alternatively, the wetness indicating composition forming a wetness indicator 15 can be applied in various other shapes (e.g., squares, circles, triangles, etc.), patterns, or incorporated into various graphic schemes of the absorbent article 10.

The wetness indicator 15 can provide a visual cue to a user and/or wearer that the absorbent article 10 has been wetted. Such a visual cue can allow the user and/or wearer to change the absorbent article 10 when necessary, and avoid disruptive checking of the absorbent article 10 for wetness. As described above, the wetness indicating composition of the wetness indicator 15 can change from a first hue to a second hue to provide such a color change. As discussed above, in some embodiments, the first hue can be blue, and the second hue can be red, pink, or purple.

Fastening System:

In an embodiment, the absorbent article 10 can include a fastening system 90. The fastening system 90 can include one or more back fasteners 91 and one or more front fasteners 92. The embodiment shown in FIGS. 1 and 2 depict an embodiment with one front fastener 92. Portions of the fastening system 90may be included in the front waist region 12, rear waist region 14, or both.

The fastening system 90 can be configured to secure the absorbent article 10 about the waist of the wearer in a fastened condition and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIGS. 1 and 2.

### Embodiments

Embodiment 1: A wetness indicating composition comprising: a mixing solvent comprising water and a water-miscible volatile organic solvent; amphiphilic polydiacetylene; and a hydrophobic binder.

Embodiment 2: The composition of embodiment 1, wherein the hydrophobic binder is selected from the group consisting of: cellulose derivatives, homopolymers and copolymers of polyacrylates, polyurethanes, and polyesters.

Embodiment 3: The composition of embodiment 1 or 2, wherein the water-miscible volatile organic solvent is selected from the group consisting of: methanol, ethanol, propanol, acetone, butanone, and tetrahydrofuran.

Embodiment 4: The composition of any one of the preceding embodiments, wherein the composition further comprises at least one of a stabilizer, a wettability modifier, and an adhesion promoter.

Embodiment 5: The composition of any one of the preceding embodiments, wherein water comprises at least 60% by weight of the mixing solvent.

Embodiment 6: The composition of any one of the preceding embodiments, wherein the composition is applied to a hydrophobic surface of a substrate with a surface tension of less than 45 dynes/cm.

Embodiment 7: The composition of any one of the preceding embodiments, wherein the composition is configured to undergo a color change from blue to a second hue upon being wetted, the second hue being selected from the group consisting of: pink and purple.

Embodiment 8: A substrate for indicating wetness, the substrate comprising: a basesheet; and a wetness indicating composition applied to a hydrophobic surface of the basesheet, the wetness indicating composition comprising: a mixing solvent comprising water and a water-miscible volatile organic solvent; amphiphilic polydiacetylene; and a hydrophobic binder.

Embodiment 9: The substrate of embodiment 8, wherein the hydrophobic binder is selected from the group consisting of: cellulose derivatives, homopolymers and copolymers of polyacrylates, polyurethanes, and polyesters.

Embodiment 10: The substrate of embodiment 8 or 9, wherein the water-miscible volatile organic solvent is selected from the group consisting of: methanol, ethanol, propanol, acetone, butanone, and tetrahydrofuran.

Embodiment 11: The substrate of any one of embodiments 8-10, wherein the mixing solvent comprises at least 60% water by weight of the mixing solvent.

Embodiment 12: The substrate of any one of embodiments 8-11, wherein the substrate is incorporated into an absorbent article.

Embodiment 13: The substrate of embodiment 12, wherein the absorbent article comprises: a body-side liner; an outer cover; and an absorbent body, the absorbent body being disposed between the body-side liner and the outer cover; wherein the hydrophobic surface of the basesheet including the wetness indicating composition is a surface of the outer cover.

Embodiment 14: The substrate of embodiment 13, wherein the outer cover comprises a facing layer and a film layer, and wherein the film layer comprises the hydrophobic surface including the wetness indicating composition.

Embodiment 15: The substrate of embodiment 14, wherein the hydrophobic surface including the wetness indicating composition is a body facing surface of the film layer.

Embodiment 16: The substrate of embodiment 12, wherein the absorbent article further comprises a fluid transfer layer, the fluid transfer layer at least partially enveloping the absorbent body, wherein the hydrophobic surface including the wetness indicating composition is a portion of the fluid transfer layer disposed between the absorbent body and the outer cover.

Embodiment 17: A method of indicating wetness in an absorbent article, the absorbent article comprising a body-side liner, an outer cover, and an absorbent body disposed between the body-side liner and the outer cover, the method comprising: providing the absorbent article, wherein the absorbent article comprises a hydrophobic surface; and applying a wetness indicating composition to the hydrophobic surface, the wetness indicating composition comprising: a mixing solvent comprising water and a water-miscible volatile organic solvent; amphiphilic polydiacetylene; and a hydrophobic binder; wherein the wetness indicating composition is configured to change color from a first hue to a second hue upon being wetted to indicate wetness in the absorbent article.

Embodiment 18: The method of embodiment 17, wherein the absorbent article further comprises a fluid transfer layer, the fluid transfer layer at least partially enveloping the absorbent body, wherein the hydrophobic surface including the wetness indicating composition is one of the outer cover and a portion of the fluid transfer layer disposed between the absorbent body and the outer cover.

Embodiment 19: The method of embodiment 17 or 18, wherein the first hue is blue and the second hue is selected from the group consisting of: pink and purple.

Embodiment 20: The method of any one of embodiments 17-19, wherein the hydrophobic binder is selected from the group consisting of: cellulose derivatives, homopolymers and copolymers of polyacrylates, polyurethanes, and polyesters.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A wetness indicating composition comprising:
a mixing solvent comprising water and a water-miscible volatile organic solvent;
amphiphilic polydiacetylene; and
a hydrophobic binder.

2. The composition of claim 1, wherein the hydrophobic binder is selected from the group consisting of: cellulose derivatives, homopolymers and copolymers of polyacrylates, polyurethanes, and polyesters.

3. The composition of claim 1, wherein the water-miscible volatile organic solvent is selected from the group consisting of: methanol, ethanol, propanol, acetone, butanone, and tetrahydrofuran.

4. The composition of claim 1, wherein the composition further comprises at least one of a stabilizer, a wettability modifier, and an adhesion promoter.

5. The composition of claim 1, wherein water comprises at least 60% by weight of the mixing solvent.

6. The composition of claim 1, wherein the composition is applied to a hydrophobic surface of a substrate with a surface tension of less than 45 dynes/cm.

7. The composition of claim 1, wherein the composition is configured to undergo a color change from blue to a second hue upon being wetted, the second hue being selected from the group consisting of: pink and purple.

8. A substrate for indicating wetness, the substrate comprising:
a basesheet; and
a wetness indicating composition applied to a hydrophobic surface of the basesheet, the wetness indicating composition comprising:
a mixing solvent comprising water and a water-miscible volatile organic solvent;
amphiphilic polydiacetylene; and
a hydrophobic binder.

9. The substrate of claim 8, wherein the hydrophobic binder is selected from the group consisting of: cellulose derivatives, homopolymers and copolymers of polyacrylates, polyurethanes, and polyesters.

10. The substrate of claim 8, wherein the water-miscible volatile organic solvent is selected from the group consisting of: methanol, ethanol, propanol, acetone, butanone, and tetrahydrofuran.

11. The substrate of claim 8, wherein the mixing solvent comprises at least 60% water by weight of the mixing solvent.

12. The substrate of claim 8, wherein the substrate is incorporated into an absorbent article.

13. The substrate of claim 12, wherein the absorbent article comprises:
a body-side liner;
an outer cover; and
an absorbent body, the absorbent body being disposed between the body-side liner and the outer cover;
wherein the hydrophobic surface of the basesheet including the wetness indicating composition is a surface of the outer cover.

14. The substrate of claim 13, wherein the outer cover comprises a facing layer and a film layer, and wherein the film layer comprises the hydrophobic surface including the wetness indicating composition.

15. The substrate of claim 14, wherein the hydrophobic surface including the wetness indicating composition is a body facing surface of the film layer.

16. The substrate of claim 12, wherein the absorbent article further comprises a fluid transfer layer, the fluid transfer layer at least partially enveloping the absorbent body, wherein the hydrophobic surface including the wetness indicating composition is a portion of the fluid transfer layer disposed between the absorbent body and the outer cover.

17. A method of indicating wetness in an absorbent article, the absorbent article comprising a body-side liner, an outer cover, and an absorbent body disposed between the body-side liner and the outer cover, the method comprising:
providing the absorbent article, wherein the absorbent article comprises a hydrophobic surface; and
applying a wetness indicating composition to the hydrophobic surface, the wetness indicating composition comprising:
a mixing solvent comprising water and a water-miscible volatile organic solvent;
amphiphilic polydiacetylene; and
a hydrophobic binder;
wherein the wetness indicating composition is configured to change color from a first hue to a second hue upon being wetted to indicate wetness in the absorbent article.

18. The method of claim 17, wherein the absorbent article further comprises a fluid transfer layer, the fluid transfer layer at least partially enveloping the absorbent body, wherein the hydrophobic surface including the wetness indicating composition is one of the outer cover and a portion of the fluid transfer layer disposed between the absorbent body and the outer cover.

19. The method of claim 17, wherein the first hue is blue and the second hue is selected from the group consisting of: pink and purple.

20. The method of claim 17, wherein the hydrophobic binder is selected from the group consisting of: cellulose derivatives, homopolymers and copolymers of polyacrylates, polyurethanes, and polyesters.
